(19) United States
(12) Patent Application Publication
(10) Pub. No.: US 2024/0401092 A1
(43) Pub. Date: Dec. 5, 2024

(54) METHOD OF PRODUCING ANTIBIOTIC- AND INDUCER-FREE VECTOR FOR PRODUCTION OF BIO-BASED MATERIALS

(71) Applicant: NATIONAL CHUNG HSING UNIVERSITY, Taichung City (TW)

(72) Inventors: Si-Yu Li, Taichung City (TW); Ying-Cheng Liao, Taichung City (TW); Boonyawee Saengsawang, Taichung City (TW); Jun-Wei Chen, Taichung City (TW); Xiao-zhen Zhuo, Taichung City (TW)

(21) Appl. No.: 18/205,132

(22) Filed: Jun. 2, 2023

(51) Int. Cl. C12P 7/625; C12N 15/70

(52) U.S. Cl. CPC C12P 7/625; C12N 15/70; C12N 2800/101

(57) ABSTRACT

The present invention mainly provides a method of producing an antibiotic- and inducer-free vector for production of bio-based materials, comprising: (a) providing a plasmid cloning vector; (b) introducing a plasmid maintenance system into the plasmid cloning vector; (c) introducing a synthetic operon into the plasmid cloning vector; and (d) introducing a ρ factor-independent terminator in the upstream of synthetic operon.

### FIELD OF INVENTION

The present invention relates to a method of producing an antibiotic-free vector for production of bio-based materials. In particular, it relates to the method of producing an antibiotic- and inducer-free vector for production of polyhydroxybutyrate.

### STATEMENT REGARDING SEQUENCE LISTING

The sequence listing associated with this application is provided in text format in lieu of a paper copy and is hereby incorporated by reference into the specification. The name of the XML file containing the sequence listing is sl.xml. The XML file is 9,940 bytes; was created on Jun. 2, 2023; and is being submitted electronically via EFS-Web.

### BACKGROUND OF THE INVENTION

Plasmid-based genetic manipulation is a common approach in metabolic engineering or whole-cell bio-catalysis. One advantage of employing plasmid-based genetic manipulation is the permit of in vitro genetic manuscript. The maintenance of recombinant plasmids in bacterial cells usually depends on the use of selective conditions, such as antibiotics supplementations. However, the scaling up of antibiotics use is expensive.

Strategies for the maintenance of plasmids during cell growth under antibiotic-free conditions have been extensively studied. Among those strategies, post-segregational killing (PSK) and active partition systems have been proven to increase plasmid stability. The hok/sok (formerly parB) locus, following the PSK mechanism, is a toxin-antidote system that simultaneously produces a toxin (Hok) and a short-lived antidote (Sok), such that in the event of plasmid loss, the cell will be killed by the long-lived toxin. In other words, only the cell that harbors the hok/sok locus can propagate because of Sok production.

Poly-3-hydroxybutyrate (PHB) has attracted much attention owing its biodegradability and biocompatibility, particularly because of the increase in global environmental concerns in recent years. The common method for PHB biosynthesis from acetyl-CoA in microorganisms comprises three steps. First, two molecules of acetyl-CoA are condensed by β-ketothiolase, encoded by phaA gene, to form acetoacetyl-CoA. Second, acetoacetyl-CoA reductase (encoded by phaB) converts acetoacetyl-CoA to 3-hydroxybutyryl-CoA using NADPH. Finally, the enzyme PHA synthase (encoded by phaC) polymerizes 3-hydroxybutyryl-CoA monomers to PHB, liberating CoA.

The above information disclosed in this section is only for enhancement of understanding of the background of the described technology and therefore it may contain information that does not form the prior art that is already known to a person of ordinary skill in the art.

### SUMMARY OF THE INVENTION

In the present invention, three pUC19-based vectors, pVEC01, pVEC02, and pVEC03, were constructed and contain alp7, hok/sok, and the alp7-hok/sok combination, respectively. The stability of pVEC01-03 without the ampicillin supplementation was tested in E. coli strains. The most stable vector among the three was chosen for application in PHB production. The putative phaCAB operon of C. manganoxidans was first amplified from the C. manganoxidans genome, where the amplicon (3,919 bp in total) contained the phaCAB gene cluster and a 194-bp fragment upstream of phaC. In this manner, the expression of phaCAB genes will be under the control of the promoter and ribosomal binding site (RBS) from C. manganoxidans. This is practically important because any possible inducer, such as IPTG, can be avoided for the economics purpose. While the phaCAB operon has been shown to be functionalized in E. coli in the present invention, the function of the recombinant plasmid containing the putative phaCAB operon in E. coli can only be shown with ampicillin supplementation. The latter part of the present invention provides a solution to take advantage of the hok/sok for antibiotic-free PHB production.

To achieve aforementioned effect, the present invention provides a method of producing an antibiotic- and inducer-free vector for production of bio-based materials, comprising: (a) providing a plasmid cloning vector; (b) introducing a plasmid maintenance system into the plasmid cloning vector; (c) introducing a synthetic operon into the plasmid cloning vector; and (d) introducing a p factor-independent terminator in the downstream of synthetic operon.

In one embodiment of the present invention, the plasmid cloning vector has pUC19.

In one embodiment of the present invention, the plasmid maintenance system comprises hok/sok system.

In one embodiment of the present invention, the ρ factor-independent terminator comprises thrLABC.

In one embodiment of the present invention, the ρ factor-independent terminator is introduced between the synthetic operon and the plasmid maintenance system.

In one embodiment of the present invention, the bio-based materials comprise polyhydroxybutyrate or poly(3-hydroxybutyrate-co-3-hydroxyvalerate).

In one embodiment of the present invention, the synthetic operon comprises phaCAB operon.

Moreover, the present invention also provides a method of producing a recombinant microorganism for production of bio-based materials, comprising introducing the aforementioned antibiotic- and inducer-free vector into the recombinant microorganism.

In one embodiment of the present invention, wherein the recombinant microorganism is E. coli or E. coli knocking out Δzwf, ΔldhA, and Δfrd.

Many of the attendant features and advantages of the present invention will become better understood with reference to the following detailed description considered in connection with the accompanying drawings.

### BRIEF DESCRIPTION OF THE DRAWINGS

The detailed structure, operating principle and effects of the present invention will now be described in more details hereinafter with reference to the accompanying drawings that show various embodiments of the present invention as follows.

FIG. 1 is a schematic diagram of the construction of pVEC01, pVEC02, pVEC03, and pPHB01 in the present invention.

FIG. 2 is the plasmid stability test of pUC19, pVEC01, pVEC02, and pVEC03 in E. coli strains (A) DH5α and (B) BL21 (DE3). Errors represent standard deviation with n=3.

FIG. 3 is (A) Glucose consumption, (B) PHB concentration, (C) PHB yield, and (D) PHB content of E. coli BL21 (DE3)/pPHB01 with and without ampicillin supplementation. The initial glucose concentration for conditions of with and without amp were 19.6±0.4 and 18.7±0.5 g/L, respectively. Errors represent standard deviation with n=3.

FIG. 4 is nile red stain assay of (A) E. coli BL21 (DE3), (B) E. coli BL21 (DE3)/pPHB01, and (C) E. coli BL21 (DE3)/pPHB01-1. The LB agar plate contained 10 g/L glucose and 0.8 mg/mL nile red without ampicillin. The plate on the left and right in each panel was taken under white light and monochromatic light at 470 nm, respectively.

FIG. 5 is maps of (A) pPHB01 and (B) pPHB01-1, where pPHB01-1 was constructed by inserting a 57-bp E. coli ρ factor-independent terminator (thrLABC, ECK120033737) into the pPHB01 to isolate the phaCAB operon and the hok/sok system.

FIG. 6 is (A) Glucose consumption, (B) PHB concentration, (C) PHB yield, and

(D) PHB content of E. coli BL21 (DE3)/pPHB01-1 with out and with 100 μg/mL ampicillin. The initial glucose concentration for conditions of with and without amp were 13±3 and 12±3 g/L, respectively. Errors represent standard deviation with n=3.

FIG. 7 is (A) OD₆₀₀ and (B) percentage of fluorescent colony forming unit (CFU) of E. coli BL21 (DE3)/pPHB01-1 for the long-term PHB production. Errors represent standard deviation with n=3.

### DETAILED DESCRIPTION OF THE PREFERRED EMBODIMENTS

Reference will now be made in detail to the exemplary embodiments of the present invention, examples of which are illustrated in the accompanying drawings. Therefore, it is to be understood that the foregoing is illustrative of exemplary embodiments and is not to be construed as limited to the specific embodiments disclosed, and that modifications to the disclosed exemplary embodiments, as well as other exemplary embodiments, are intended to be included within the scope of the appended claims. These embodiments are provided so that this invention will be thorough and complete, and will fully convey the inventive concept to those skilled in the art.

For convenience, certain terms employed in the specification, examples and appended claims are collected here. Unless defined otherwise, all technical and scientific terms used herein have the same meaning as commonly understood by one of the ordinary skill in the art to which this invention belongs.

Various embodiments will now be described more fully with reference to the accompanying drawings, in which illustrative embodiments are shown. The inventive concept, however, may be embodied in various different forms, and should not be construed as being limited only to the illustrated embodiments. Rather, these embodiments are provided as examples, to convey the inventive concept to one skilled in the art. Accordingly, known processes, components, and techniques are not described with respect to some of the embodiments.

The singular forms “a”, “and”, and “the” are used herein to include plural referents unless the context clearly dictates otherwise.

The biological materials used in the present invention need not be deposited according to 37 CFR 1.802 since those biological materials are known and available to the public, or can be made, or isolated without undue experimentation.

In the present invention, an antibiotic- and inducer-free culture condition was proposed for bio-based materials, such as polyhydroxybutyrate (PHB), production in recombinant Escherichia coli. First, antibiotic-free vectors were constructed by installing the plasmid maintenance system, alp7, hok/sok, and the hok/sok and alp7 combination into the pUC19 vector. The plasmid stability test showed that pVEC02, the pUC19 vector containing the hok/sok system, was the most effective in achieving antibiotic-free cultivation in the E. coli. Second, the putative phuCAB operon derived from Caldimonas manganoxidans was inserted into pVEC02 to yield pPHB01 for PHB production in E. coli BL21 (DE3). The putative phaCAB operon was first shown function properly for PHB production and thus, inducer-free conditions were achieved. However, the maintenance of pPHB01 in E. coli requires antibiotics supplementation. Finally, an efficient E. coli ρ factor-independent terminator, thrLABC (ECK120033737), which is available from Chen et al., 2013b (Chen, Y.-J., Liu, P., Nielsen, A.a.K., Brophy, J.a.N., Clancy, K., Peterson, T., and Voigt, C. A. (2013b). Characterization of 582 natural and synthetic terminators and quantification of their design constraints. Nature Methods 10, 659-664), was inserted in the upstream of phaCAB operon, preferably inserted between the phaCAB operon and the hok/sok system. The newly constructed plasmid pPHB01-1 facilitates an antibiotic- and inducer-free culture condition and induces the production of PHB with a concentration of 3.0±0.2 g/L, yield of 0.26±0.07 g/g-glucose, and content of 44±3%. The PHB production using E. coli BL21 (DE3)/pPHB01-1 has been shown to last 84 and 96 h in the liquid and solid cultures.

It should be noted that, the product in the present invention includes, but not limited to polyhydroxybutyrate. Any bio-based materials that can be theoretically produced by the process of the present invention can be the products in the present invention. Similarly, the phaCAB operon used in the present invention is merely one kind of the synthetic operon, and any synthetic operon that is suitable for the process of the present invention can be employed in the present invention.

The following descriptions are provided to elucidate a method of producing an antibiotic- and inducer-free vector for production of bio-based materials and to aid it of skilled in the art in practicing this invention. These embodiments are merely exemplary embodiments and in no way to be considered to limit the scope of the invention in any manner.

### Material and Method

### Bacterial Strains and Plasmids

All the bacterial strains and plasmids used in the present invention, and the sources are listed in Table 1 as follows.

TABLE 1
List of bacterial strains and plasmids used in the present invention

| Name | Descriptions | Reference |
|---|---|---|
| Bacterial strains | | |
| E. coli DH5α | F⁻ endA1 glnV44 thi-1 recA1 relAl gyrA96 deoR | Available |
| | nupG purB20 φ80dlacZΔM15 Δ(lacZYA-argF)U169, | from BCRC |
| | hsdR17(rK⁻mK⁺), λ⁻ | |
| E. coli BL21 (DE3) | F⁻, dcm, ompT, gal, lon, hsdS_{B}(rB⁻, mB⁻), | Available |
| | λ(DE3[lacI, lacUV5-T7 gene 1, ind1, sam7, nin5]) | from BCRC |
| E. coli MG1655 | F⁻ lambda⁻ ilvG- rfb-50 rph-1 | Available |
| | | from BCRC |
| E. coli MZLF | E. coli BL21 (DE3) Δzwf, ΔldhA, Δfrd | Yang et |
| | | al., 2016 |
| C. manganoxidans | JCM 10698 (BCRC 17858) | Available |
| | | from BCRC |
| Plasmids | | |
| pUC19 | Commonly used cloning vector that conveys Tet and | Available |
| | Amp resistance. | from TBC |
| pTKW106alp7A | Recombinant plasmid carries alp7CAR gene and | Danino et |
| | hok/sok gene | al., 2015 |
| pVEC01 | Recombinant plasmid carries alp7CAR gene (derived | The present |
| | from B. subtilis pLS20) to maintain the plasmid | invention |
| | stability. | |
| pVEC02 | Recombinant plasmid carries hok/sok gene (derived | The present |
| | from E. coli R1 plasmid) to maintain the plasmid | invention |
| | stability. | |
| pVEC03 | Recombinant plasmid carries alp7CAR gene and | The present |
| | hok/sok gene to maintain the plasmid stability. | invention |
| pPHB01 | pVEC02-based plasmid carries hok/sok gene and | The present |
| | phaCAB operon (derived from C. manganoxidans) | invention |
| | under the control of the native promoter of phaCAB | |
| | operon from C. manganoxidans. | |
| pPHB01-1 | pPHB01 derived recombinant plasmid where a | The present |
| | terminator was inserted between hok/sok gene and | invention |
| | phaCAB operon | |

1. BCRC is Bioresource Collection and Research Center, Taiwan.
2. TBC is Taigen bioscience corp., Taiwan
3. E. coli MZLF was available from Yang et. al. (C.-H. Yang, E.-J. Liu, Y.-L. Chen, F.-Y. Ou-Yang and S.-Y. Li, Microbial cell factories, 2016, 15, 133-133.)
4. Plasmids pTKW106alp7A was available from Danino et al. (Danino, T., Prindle, A., Kwong, G. A., Skalak, M., Li, H., Allen, K., Hasty, J., and Bhatia, S. N. (2015)).

### DNA Manipulation and Transformation

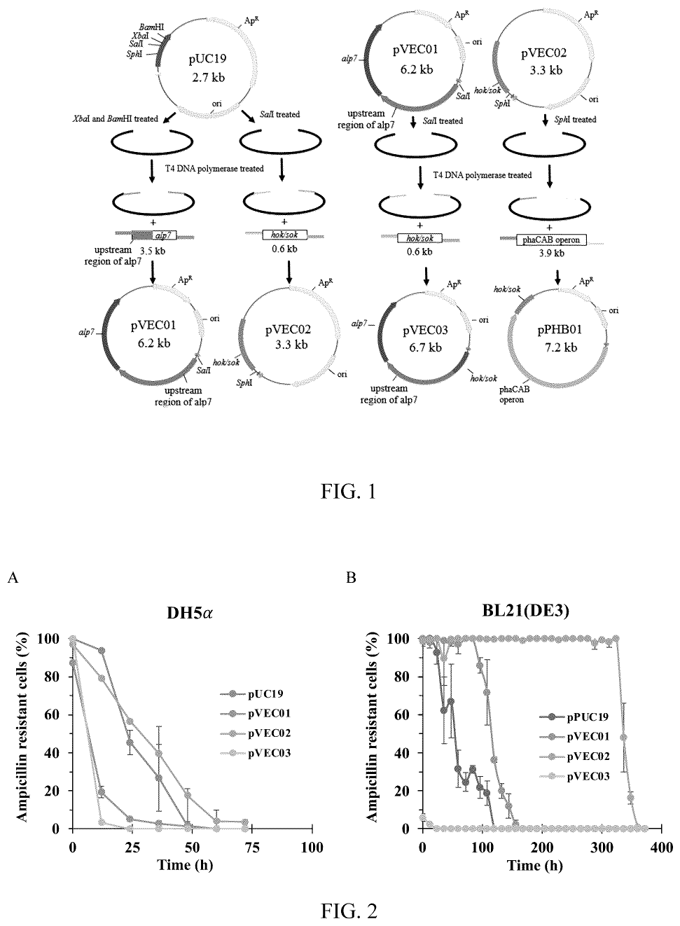

Plasmids were constructed by sequence- and ligation-independent cloning (Li, M. Z., and Elledge, S. J. (2007). Nature methods 4, 251; Jeong, J.-Y., Yim, H.-S., Ryu, J.-Y., Lee, H. S., Lee, J.-H., Seen, D.-S., and Kang, S. G. (2012). Appl. Environ. Microbiol. 78, 5440-5443; Islam, M. N., Lee, K. W., Yim, H.-S., Lee, S. H., Jung, H. C., Lee, J.-H., and Jeong, J.-Y. (2017). BioTechniques 63, 125-130.) and schematic diagrams of plasmid construction are shown in FIG. 1. The primers used in the present invention are listed in Table 2. To construct the recombinant plasmid pVEC01, alp7 was amplified from pTKW106alp7 (Addgene, Watertown, USA) with the primer pair SLIC-F-alp7-I (SEQ. ID. NO. 1) and SLIC-R-alp7-i (SEQ. ID. NO. 2) by polymerase chain reaction (PCR), and pUC19 was digested with XbaI and BamHI. To construct the recombinant plasmids pVEC02 and pVEC03, the hok/sok locus was amplified from pTKW106alp7 with the primer pair SLIC-F-parB-01 (SEQ. ID. NO. 3) and SLIC-R-parB-01 (SEQ. ID. NO. 4) by PCR, and the vectors pUC19 and pVEC01 were digested with SalI. To construct the recombinant plasmid pPHB01, the phaCAB operon was amplified from the genomic DNA of C. manganoxidans with the primer pair SLIC-F-phaCAB1-01 (SEQ. ID. NO. 5) and SLIC-R-phaCAB1-01 (SEQ. ID. NO. 6), and pVEC02 was digested with SphI. After the preparation of inserts and vectors, the DNA fragments were purified using the Gene-Spin™ 1-4-3 DNA Extraction Kit (PROTECH, Taipei, Taiwan). The insert and corresponding vector were mixed at an appropriate ratio, which is around 5:1˜1:5, and incubated at 37° C. for 1 min with T4 DNA polymerase to generate 5′-overhangs. Thereafter, the reaction mixture was placed on ice for 20 min for single-strand annealing and was introduced into competent E. coli DH5α cells for transformation.

To construct the recombinant plasmid pPHB01-1, the Q5® site-directed mutagenesis kit (NEB, Ipswich, USA) was used to insert a 57-bp E. coli ρ factor-independent terminator, thrLABC (ECK120033737) between the phaCAB operon and the hok/sok locus in pPHB01. The primers used for constructing pPHB01-1 were In/del-F-pPHB01-1 (SEQ. ID. NO. 7) and In/del-R-pPHB01-1 (SEQ. ID. NO. 8). After PCR, the amplified DNA was treated with a Kinase-Ligase-DpnI enzyme mix at room temperature for 5 min for circularization and template removal. Mutant selection was enriched by simultaneous ligation and DpnI treatment. Next, 5 μL of the KLD mix was directly introduced into competent E. coli DH5α cells. Colony PCR was used for pPHB01-1 screening with the primers CK-F-pPHB01-1 (in-del) (SEQ. ID. NO. 9) and CK-R-pPHB01-1 (in-del) (SEQ. ID. NO. 10). Finally, pPHB01-1 was confirmed by DNA sequencing. All recombinant plasmids constructed in the present invention were verified by colony PCR, restriction enzyme digestion, and DNA sequencing.

TABLE 2
Primer sequence in the
present invention

| | | SEQ ID |
|---|---|---|
| Primer | Sequence | NO: |
| SLIC-F-alp7-i | tgcctgcagg | (1) |
| | tcgactctag | |
| | accacctagg | |
| | tcattagcct | |
| SLIC-R-alp7-i | gagctcggta | (2) |
| | cccggggatc | |
| | tcagggcgtc | |
| | tgtgttgcaa | |
| SLIC-F-parB-01 | cttgcatgcc | (3) |
| | tgcaggtcga | |
| | aacaaactcc | |
| | gggaggcagc | |
| SLIC-R-parB-01 | cctaggtggt | (4) |
| | ctagagtcga | |
| | acaacatcag | |
| | caaggagaaa | |
| SLIC-F-phaCAB1-01 | ccatgattac | (5) |
| | gccaagcttg | |
| | ggtacatgga | |
| | gcagatgcgc | |
| SLIC-R-phaCAB1-01 | agtttgtttc | (6) |
| | gacctgcagg | |
| | cgagttgatc | |
| | gccaacgaag | |
| In/del-F-pPHB01-1 | cccgcacctg | (7) |
| | acagtgcggg | |
| | cttttttttt | |
| | cgaccaaagg | |
| | agtgccgctc | |
| | ttcgttggcg | |
| | atcaactcg | |
| In/del-R-pPHB01-1 | cttttttctg | (8) |
| | tgtttccatt | |
| | gttagacgag | |
| | agtgtgctca | |
| | gttgtcaagc | |
| | cgtccgccgg | |
| | gacggccgca | |
| | tgtcctggct | |
| | tac | |
| CK-F-pPHB01-1 | gaagaaatcg | (9) |
| (in-del) | cttcgatcg | |
| CK-R-pPHB01-1 | aaaccacctt | (10) |
| (in-del) | cacgtcatg | |

### Plasmid Stability Test

Escherichia coli strains DH5α, MG1655, BL21 (DE3), and MZLF were used as hosts to test plasmid stability. MZLF is obtained from Yang et. al. (C.-H. Yang, E.-J. Liu, Y.-L. Chen, F.-Y. Ou-Yang and S.-Y. Li, Microbial cell factories, 2016, 15, 133-133.). Cells for the plasmid stability test were grown from −80° C. stocks by streaking on an LB plate with ampicillin. After 16-24 h, a single colony was picked and used to inoculate LB containing antibiotics for 12 h. Then, 10 μL of the cell suspensions was inoculated into 3 mL LB without antibiotics to start the stability experiment. The inoculum was diluted and plated on LB plates supplemented with or without antibiotics.

After 12 h, 10 μL of the cell suspensions was transferred to a new test tube. Every 12 h, 10 μL of the sample was transferred to a new flask, and the cells were diluted and plated on LB plates supplemented with or without antibiotics. Plasmid stability (segregation) was estimated by the equation:

$$Ampicillin - resistant cells ( \% ) = \frac{colonies on LB + ampicillin}{colonies on LB} \times 100 \%$$

Characterization of the Putative Promoter of the phaCAB Operon from C. manganoxidans.

The putative promoter of the phaCAB operon was predicted by BPROM (http://www.softberry.com/berry.phtml) and putative ribosome binding sites (RBSs) were reported in a previous study (Lin, J.-H., Lee, M.-C., Sue, Y.-S., Liu, Y.-C., and Li, S.-Y. (2017), Applied microbiology and biotechnology 101, 6419-6430).

### Culture Conditions for PHB Production

The strains used for shake flask experiments were grown aerobically at 200 rpm and 37° C. in fresh 25-mL LB medium supplemented with 20 g/L glucose, 0.6 g/L MgSO₄·7H₂O, 0.07 g/L CaCl₂·2H₂O, 0.04 g/L KH₂PO₄, 0.04 g/L K₂HPO₄, 0.4 g/L NaHCO₃ and 100 μg/mL ampicillin. In the shake flask experiments, the initial OD₆₀₀ was adjusted to 0.05. Samples were taken and stored every 12 h until 48 h for the subsequent analysis.

### Analytical Methods

Cell density was monitored by measuring the optical density at 600 nm (GENESYS 10S, Thermo Scientific, USA). For the measurement of biomass concentration, 3 mL of the cultured cells were centrifuged for 1 min at 6,791 rcf. The cells were frozen at −20° C. and lyophilized. After freeze-drying, the weight of the sample was recorded to calculate the biomass concentration. The PHB produced by the recombinant E. coli cells was quantified by gas chromatography (GC). Briefly, an appropriate amount of biomass was transferred to a clean spiral test tube and 1 mL of chloroform, 0.85 mL of methanol, and 0.15 mL of sulfuric acid were added. The tube was incubated in a water bath for 140 min at 80° C. and cooled down to room temperature. Then 1 mL of DI H₂O was mixed well by vortexing. After standing and layering, the organic phase was removed and filtered through a 0.2-μm PVDF filter and then analyzed by GC. The temperatures of the injector and detector were 230° C. and 275° C., respectively. The temperature of the column was set at 100° C. and increased to 200° C. at a rate of 10° C./min and maintained at 200° C. for 2 min. The nitrogen was used as the carrier gas at 3 mL/min. The split mode with the split ratio of 1:10 was used.

The metabolites were determined using a Thermo Scientific™ Dionex™ Ultimate 3000 LC system equipped with an ORH-801 column. Sulfuric acid (5 mM) was used as the mobile phase, and the flow rate was maintained at 0.6 mL/min. Samples for quantification were collected from the culture media after the removal of the cells by centrifugation for 1 min at 6,791 rcf. The supernatant was passed through a 0.2-μm PVDF filter, and the sample injection (10 μL) was performed using an auto-sampler.

### Sodium Dodecyl Sulphate-Polyacrylamide Gel Electrophoresis (SDS-PAGE)

The bacterial culture solutions were centrifuged at 4° C. and 9,072×g for 10 min and resuspended in lysis buffer (50 mM Tris, 0.1 M NaCl, 1 mM EDTA, pH 7.8) to obtain a final OD of 20. The concentrated bacterial solutions were subjected to ultrasonication (10 s break and 5 s intermittent, total 60 cycle) (QSonica Q700, USA). The cell lysates were then diluted with 4× Laemmli sample buffer (Bio-Rad Laboratories, Inc, USA) and heated in a dry batch for 10 min. Samples (10 μL) were loaded into the wells for SDS-PAGE analysis (12% acrylamide).

### Nile Red Stain Assay

To confirm in vivo PHB production, the recombinant E. coli BL21 (DE3)/pPHB01 was grown on LB plates containing 10 g/L glucose and 0.8 mg/mL nile red. After 24 h of cultivation at 37° C., the plates were exposed to monochromatic light at 470 nm for observation.

### Results

Stability of pVEC01, pVEC02, and pVEC03 in E. coli

The recombinant plasmids pVEC01, pVEC02, and pVEC03 were tested for plasmid stability in different E. coli hosts. Compared to the parental plasmid pUC19, pVEC02 showed good stability in E. coli DH5α, E. coli BL21 (DE3) and with time required to reach 50% ampicillin-resistant cells of 26 and 336 h, respectively (while pUC19 had times of 25 and 54 h, respectively, FIGS. 2A and 2B). The hok/sok system increased the segregational stability in BL21 (DE3) by 6 fold. pVEC01 was slightly more stable than pUC19 in BL21 (DE3) cells and pVEC03 showed instability. Because of the stability of pVEC02 in this embodiment, it was chosen as the new vector for the subsequent induction of PHB production.

PHB Production by E. coli BL21 (DE3)/pPHB01 in Shake Flasks

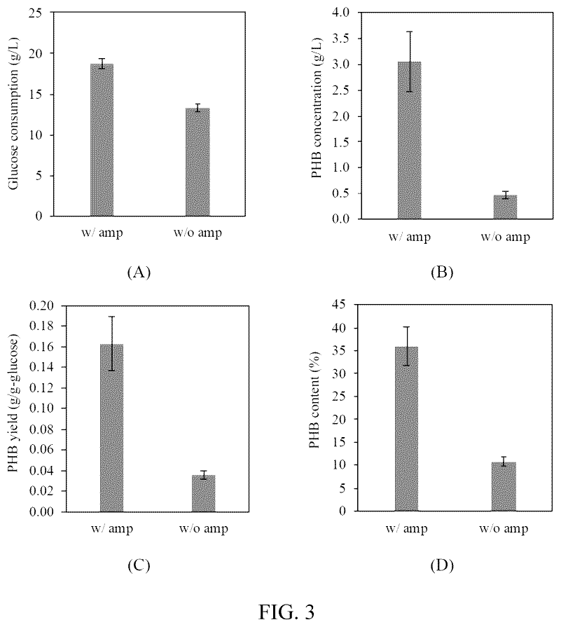

PHB production is tested by conducting shake flask experiments at 37° C. under the control of the native promoter of phaCAB operon from C. manganoxidans. FIG. 3A shows that E. coli BL21 (DE3)/pPHB01 consumed 18±1 and 13±1 g/L glucose with and without ampicillin, respectively. In the presence of ampicillin, E. coli BL21 (DE3) harboring pPHB01 was able to effectively produce PHB and achieved a PHB concentration of 3.1±0.6 g/L (FIG. 3B), yield of 0.16±0.03 g/g-glucose, and content of 36±4% (FIG. 3D). In contrast, in the absence of ampicillin, E. coli BL21 (DE3) harboring pPHB01 produced PHB with a decreased concentration of 0.5±0.1 g/L, as shown in FIGS. 3B-3D. The effective PHB production in E. coli BL21 (DE3)/pPHB01 justifies the high glucose consumption in the presence of ampicillin.

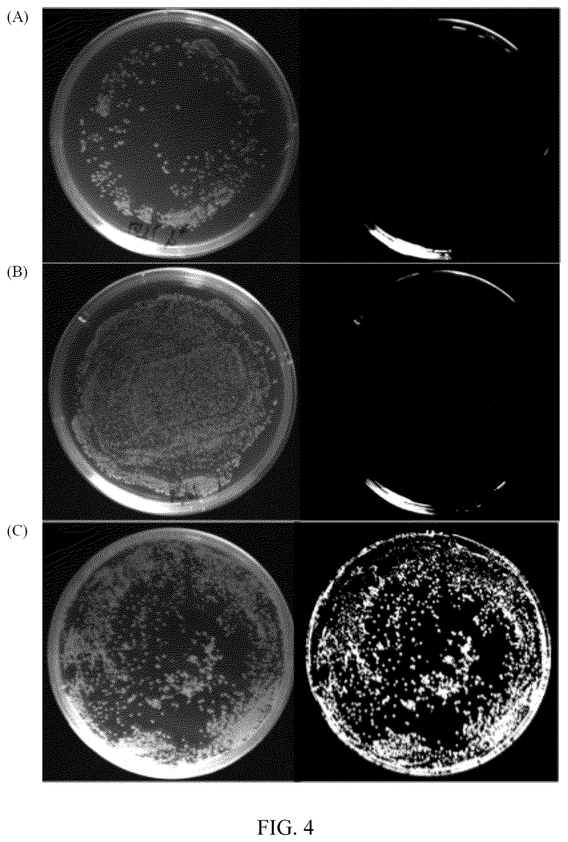

The capability of E. coli BL21 (DE3)/pPHB01-1 to produce PHB without the inducer and antibiotics was confirmed by the Nile red assay, as shown in FIG. 4.

Isolation of the phaCAB Operon and Hok/Sok Genetic Units by Inserting a Terminator

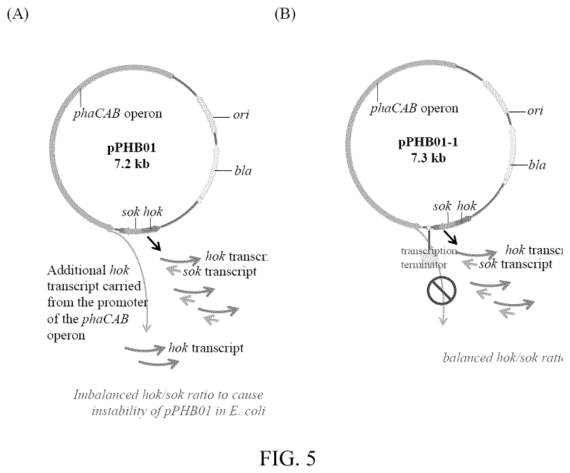

While pVEC02 (hok/sok) was shown to be a stable vector in E. coli without ampicillin supplementation, the low PHB production by E. coli BL21 (DE3)/pPHB01 in the absence of ampicillin indicates the instability of pPHB01 in E. coli BL21 (DE3). It is speculated that the insertion of the phaCAB operon in pVEC02 disrupted the balance of hok/sok transcripts. In other words, the promoter of the phaCAB operon may carry an additional transcript of hok and make pPHB01 toxic to E. coli (FIG. 5). To verify this speculation, an efficient E. coli p factor-independent terminator, thrLABC (ECK120033737), was inserted between the phaCAB operon and the hok/sok locus to isolate the two genetic units (FIG. 5). The newly constructed plasmid was named pPHB01-1. Furthermore, 5-bromo-UTP or 8-azido-ATP nucleotide may be added to improve the termination effect and PHB production in this embodiment.

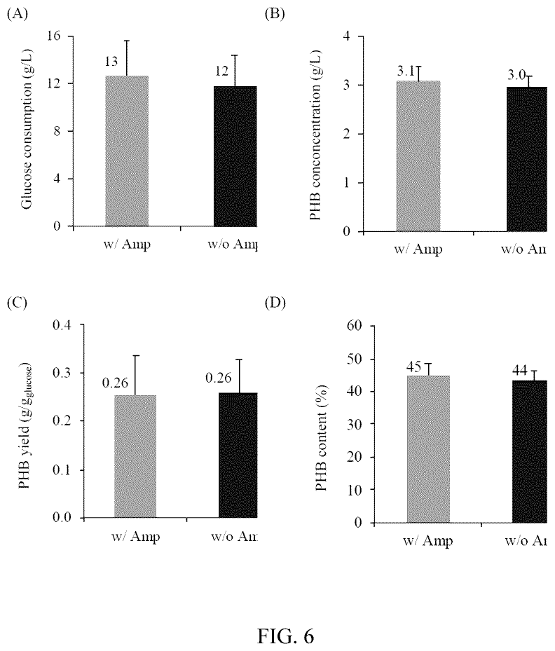

E. coli BL21 (DE3) harboring the newly constructed plasmid pPHB01-1 in was tested for the PHB production with and without ampicillin supplementation (FIG. 6). In the presence of ampicillin, E. coli BL21 (DE3)/pPHB01-1 was able to produce PHB effectively. All the fed glucose of 13±3 g/L was completely consumed in 48 h (FIG. 6A), and the PHB concentration, yield, and content were 3.1±0.3 g/L, 0.26±0.08 g/g-glucose, and 45±4%, respectively (FIGS. 6B-D). In the absence of ampicillin supplementation, E. coli BL21 (DE3)/pPHB01-1 was still able to provide a comparable PHB performance, where the PHB concentration, yield, and content was 3.0±0.2 g/L, 0.26±0.07 g/g-glucose, and 44±3%, respectively (FIGS. 6B-D).

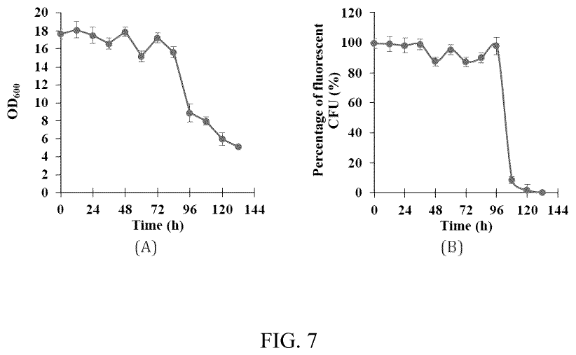

To characterize the long-term stability of PHB production in E. coli BL21 (DE3)/pPHB01-1, E. coli BL21 (DE3)/pPHB01-1 was first cultivated in the liquid culture without the ampicillin (the medium composition was described in 2.5) and transferred to a fresh medium every 12 h. During the transfer, the grown-culture solution was spread on the agar plate without the ampicillin but 0.8 mg/mL nile red. After the formation of CFU, the total CFU as well as the fluorescent CFU were counted to quantify the percentage of CFU that can produce PHB. It can be seen in FIG. 7A that the OD₆₀₀, which is positively correlated to the PHB production, can be maintained in the range of 15 and 18 in the first 84 h and gradually dropped to 5.1±0.1 at 132 h. FIG. 7B shows that the E. coli BL21 (DE3)/pPHB01-1 maintained its ability to produce PHB in the first 96 h and less than 20% of cell population can produce PHB afterwards.

It should be noted that, in this embodiment, the ρ factor-independent terminator was inserted between the phaCAB operon and the hok/sok system. However, it is not limited thereto. The ρ factor-independent terminator introduced into the upstream of phaCAB operon also works in the present invention.

### DISCUSSION

In the present invention, a high-copy number backbone, pUC19, was chosen since it may benefit recombinant protein production and metabolic engineering. PSK and active partition systems are usually found in a low-copy number plasmid. It will be interesting to see how well PSK and active partition systems maintain the high-copy pUC19 in E. coli. The present invention clearly demonstrated that alp7 system was incompatibility with the high-copy backbone. This may because the active partition of the plasmid consumes ATP during DNA replication. Energy consumption is critical for the function of alp7, and the installation of alp7 in a high-copy number backbone may consume too much ATP, making alp7/pUC19 incompatible with E. coli. In contrast, the genetic unit hok/sok in pVEC02 was found to be an efficient antibiotic-free selection marker in E. coli, including E. coli BL21 (DE3) and MZLF. The time to reach 50% ampicillin-resistant cells can go up to 360 h, which is competitive among literatures. E. coli K and B strains bear 5 and 6 hok/sok loci in the chromosome, respectively, and all of them are considered inactive yet may be induced by unknown signal.

The application of the antibiotic-free vector pVEC02 was further investigated in metabolic engineering for PHB production by constructing the recombinant pPHB01. E. coli BL21 (DE3)/pPHB01 can effectively produce PHB; however, only in the presence of antibiotics. The ineffective function of the hok/sok system in pPHB01 demonstrates that the hok/sok system in the engineering perspective needs more detailed investigation. The present invention focused on the interaction among genetic units to determine the antibiotic demand of pPHB01. It is speculated that the transcription activity of the phaCAB operon passes downstream of the hok/sok gene unit. The transcriptional carry-over increases hok transcription; therefore, the hok/sok balance is interrupted. This carry-over of the transcription activity can be especially severe when the downstream hok is a short transcript. To prevent transcriptional carry-over, an efficient E. coli ρ factor-independent terminator, thrLABC (ECK120033737), was inserted between the phaCAB operon and the hok/sok gene unit (FIG. 6). The isolation of the two genetic units was shown in the present invention to be effective in finding an application for pVEC02 in metabolic engineering. In addition, a putative promoter region of the phaCAB operon from C. manganoxidans was first shown properly while effectively function in E. coli. This is the first application to demonstrate that the phaCAB operon from C. manganoxidans can be used for effective PHB production in E. coli. In summary, the inducer-free PHB production can be achieved by adopting heterologous promoter from C. manganoxidans. The antibiotic-free PHB production involves the interplay among bacteria chassis, antibiotic-free genetic markers, and the genetic operon for PHB production. The present invention presents a right combination to achieve the inducer- and antibiotic-free system for PHB production.

It will be understood that the above description of embodiments is given by way of example only and that various modifications may be made by those with ordinary skill in the art. The above specification, examples, and data provide a complete description of the present invention and use of exemplary embodiments of the invention. Although various embodiments of the invention have been described above with a certain degree of particularity, or with reference to one or more individual embodiments, those with ordinary skill in the art could make numerous alterations or modifications to the disclosed embodiments without departing from the spirit or scope of the present invention.

What is claimed is:

1. A method of producing an antibiotic- and inducer-free vector for production of bio-based materials, comprising:
(a) providing a plasmid cloning vector;
(b) introducing a plasmid maintenance system into the plasmid cloning vector;
(c) introducing a synthetic operon into the plasmid cloning vector; and
(d) introducing a ρ factor-independent terminator in the downstream of synthetic operon.

2. The method of claim 1, wherein the plasmid cloning vector has pUC19.

3. The method of claim 1, wherein the plasmid maintenance system comprises hok/sok system.

4. The method of claim 1, wherein the ρ factor-independent terminator comprises thrLABC.

5. The method of claim 1, wherein the ρ factor-independent terminator is introduced between the synthetic operon and the plasmid maintenance system.

6. The method of claim 1, wherein the bio-based materials comprise polyhydroxybutyrate or poly(3-hydroxybutyrate-co-3-hydroxyvalerate).

7. The method of claim 1, wherein the synthetic operon comprises phaCAB operon.

8. A method of producing a recombinant microorganism for production of bio-based materials, comprising introducing an antibiotic- and inducer-free vector according to claim 1 into the recombinant microorganism.

9. The method of claim 8, wherein the recombinant microorganism has E. coli or E. coli knocking out Δzwf, ΔldhA, and Δfrd.

10. The method of claim 8, wherein the bio-based materials comprise polyhydroxybutyrate or poly(3-hydroxybutyrate-co-3-hydroxyvalerate).


SEQUENCE LISTING (10 sequences)

SEQ ID NO: 1 - DNA, 40 bp, synthetic construct
  source 1..40: mol_type = other DNA
tgcctgcaggtcgactctagaccacctaggtcattagcct

SEQ ID NO: 2 - DNA, 40 bp, synthetic construct
  source 1..40: mol_type = other DNA
gagctcggtacccggggatctcagggcgtctgtgttgcaa

SEQ ID NO: 3 - DNA, 40 bp, synthetic construct
  source 1..40: mol_type = other DNA
cttgcatgcctgcaggtcgaaacaaactccgggaggcagc

SEQ ID NO: 4 - DNA, 40 bp, synthetic construct
  source 1..40: mol_type = other DNA
cctaggtggtctagagtcgaacaacatcagcaaggagaaa

SEQ ID NO: 5 - DNA, 40 bp, synthetic construct
  source 1..40: mol_type = other DNA
ccatgattacgccaagcttgggtacatggagcagatgcgc

SEQ ID NO: 6 - DNA, 40 bp, synthetic construct
  source 1..40: mol_type = other DNA
agtttgtttcgacctgcaggcgagttgatcgccaacgaag

SEQ ID NO: 7 - DNA, 69 bp, synthetic construct
  source 1..69: mol_type = other DNA
cccgcacctgacagtgcgggctttttttttcgaccaaaggagtgccgctcttcgttggcgatcaactcg

SEQ ID NO: 8 - DNA, 83 bp, synthetic construct
  source 1..83: mol_type = other DNA
cttttttctgtgtttccattgttagacgagagtgtgctcagttgtcaagccgtccgccgggacggccgcatgtcctggcttac

SEQ ID NO: 9 - DNA, 19 bp, synthetic construct
  source 1..19: mol_type = other DNA
gaagaaatcgcttcgatcg

SEQ ID NO: 10 - DNA, 19 bp, synthetic construct
  source 1..19: mol_type = other DNA
aaaccaccttcacgtcatg